(19) United States
(12) Patent Application Publication
(10) Pub. No.: US 2007/0299317 A1
(43) Pub. Date: Dec. 27, 2007

(54) System and method for programming customized data collection for an autonomous medical device

(76) Inventors: Kenneth P. Hoyme, Plymouth, MN (US); James O. Gilkerson, Stillwater, MN (US); James R. Kalgren, Lino Lakes, MN (US)

Correspondence Address: CASCADIA INTELLECTUAL PROPERTY, 500 UNION STREET, STE.1005, SEATTLE, WA, 98101, US

(21) Appl. No.: 11/452,748

(22) Filed: Jun. 13, 2006

(51) Int. Cl. A61B 5/00

(52) U.S. Cl. 600/300

(57) ABSTRACT

A system and method for programming customized data collection for an autonomous medical device is presented. Data measures to be recorded by an autonomous medical device with remote monitoring are dynamically identified to evaluate a patient status. One or more operational parameters for the autonomous medical device that specify collection of the data measures are defined. A data source to measure each data measure is identified. Collection conditions applicable to the data source are specified. Resources allocated to transiently stage the data measure in the autonomous medical device are managed. The operational parameters are programmed to effect functional changes in the collection of the data measures to the autonomous medical device. The autonomous medical device is regularly interrogated to remotely retrieve the data measures recorded under the operational parameters.

### FIELD OF THE INVENTION

The invention relates in general to medical device management and, specifically, to a system and method for programming customized data collection for an autonomous medical device.

### BACKGROUND OF THE INVENTION

Patient medical devices are used to provide therapy and monitoring to patients most frequently suffering from chronic diseases, such as cardiopulmonary disorders, including arrhythmias and tachycardia. Implantable medical devices (IMDs) are surgically implanted in patients to provide in situ therapy and monitoring over an extended time period. External medical devices (EMDs) are frequently worn or placed proximate to patients to provide short-term care, often in a non-ambulatory setting. Both IMDs and EMDs can include sensors to monitor patient physiology and related data, which are recorded and stored temporarily for retrieval and evaluation during periodic patient follow-up.

In-clinic patient follow-up often requires specialized equipment, such as programmer recorders, to access data in and to reprogram patient medical devices, particularly IMDs. As an alternative, patient management devices (PMDs) permit limited remote patient follow-up to be completed outside of a clinical setting, such as in a patient's home. PMDs are patient-operable devices that perform functions similar to programmer recorders. Caregivers can remotely interrogate PMDs to retrieve patient data for centralized evaluation and to ensure compliance with the prescribed treatment regimens. Patients can also perform self-initiated interrogations per caregiver instructions or following an event occurrence. However, PMDs generally do not support remote programming.

Patient medical devices often have limited resources and most IMDs have tightly-constrained processing, storage, and power resources. Nevertheless, long-term patient medical devices must store up to three to twelve months of patient data between follow-up sessions. Frequently, to maximize available resource utilization, only one set of patient measures are recorded per day and are averaged weekly to compress data storage overhead. As a result, patient follow-up can be artificially restricted by the data actually captured. For instance, the monitoring features enabled by a patient medical device can potentially limit the scope of medical review and evaluation by filtering out transient but medically significant events. Thus, long-term patient monitoring through resource-limited patient medical devices strikes a compromise between the need to collect patient data between follow-up sessions and the limited resources available to support patient monitoring over an extended period.

Unfortunately, the compromise can be a trade-off through which important physiometry can potentially be lost. For instance, original recorded data measures that are subsequently compressed or averaged are irretrievably lost in the data conversion. Patient data can also be missed by a patient medical device where physiometric changes are occurring more rapidly than values are recorded or the data measures fall outside the capabilities of the patient medical device to capture. Moreover, patient data can be skipped due to a lack of memory for storage. For instance, conventional IMDs generally employ a “sliding window” storage model that allocates a fixed amount of memory and patient data is compressed, averaged, or deleted when too old to remain in the window. As well, particular types of patient data can be omitted where the patient medical device or sensors are not configured to monitor and record that data type. Finally, patient data can be wasted, such as where recorded physiometric measures are not needed for following a particular patient's health status.

Therefore, there is a need for providing ad hoc programming of operational characteristics of patient medical devices that are remotely monitored to permit flexible selection of and control over collection of patient data in light of available and limited on-board patient medical device resources.

### SUMMARY OF THE INVENTION

A system and method includes dynamically programming one or more remotely managed patient medical devices to tailor the characteristics of patient data collection, for instance, the patient data type and sampling frequency, for remotely followed patients. Patient medical devices include both IMDs and EMDs. Patient well-being and clinical trajectory are initially evaluated based on available patient data and operational parameters are defined to generate feature sets for dynamic programming into the patient medical devices. The operational parameters can include data collection schemes, including indications-based and event-triggered schemes. Following the dynamic programming, accumulated patient data is uploaded from the patient medical devices and centrally archived to allow more frequent and diverse patient data collection to occur between interrogations. In a further embodiment, overall memory usage is estimated and data collection intervals are modified as necessary to conserve the resources available until the next interrogation.

One embodiment provides a system and method for programming customized data collection for an autonomous medical device. Data measures to be recorded by an autonomous medical device with remote monitoring are dynamically identified to evaluate a patient status. One or more operational parameters for the autonomous medical device that specify collection of the data measures are defined. A data source to measure each data measure is identified. Collection conditions applicable to the data source are specified. Resources allocated to transiently stage the data measure in the autonomous medical device are managed. The operational parameters are programmed to effect functional changes in the collection of the data measures to the autonomous medical device. The autonomous medical device is regularly interrogated to remotely retrieve the data measures recorded under the operational parameters.

Still other embodiments will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a block diagram showing, by way of example, an implantable medical device for use with one embodiment.

FIG. 2 is a functional block diagram showing a system for programming customized data collection for an autonomous medical device, in accordance with one embodiment.

FIG. 3 is a set of Venn diagrams showing, by way of example, data measures as grouped before and after dynamic data collection specification through the system of FIG. 2.

FIG. 4 is a functional block diagram showing dynamic data collection specification and execution in the system of FIG. 2.

FIG. 5 is a process flow diagram showing a method for programming customized data collection for an autonomous medical device, in accordance with one embodiment.

FIG. 6 is a block diagram showing, by way of example, operational parameters for specification through the system of FIG. 2.

FIG. 7 is a flow diagram showing a routine for specifying a scheme for use in the method of FIG. 5.

FIG. 8 is a flow diagram showing a routine for defining triggers for use in the method of FIG. 5.

FIG. 9 is a flow diagram showing a routine for performing dynamic memory control for use in the method of FIG. 5.

FIG. 10 is a functional block diagram showing a data collection specifier for use in the system of FIG. 2.

### DETAILED DESCRIPTION

### Implantable Medical Device

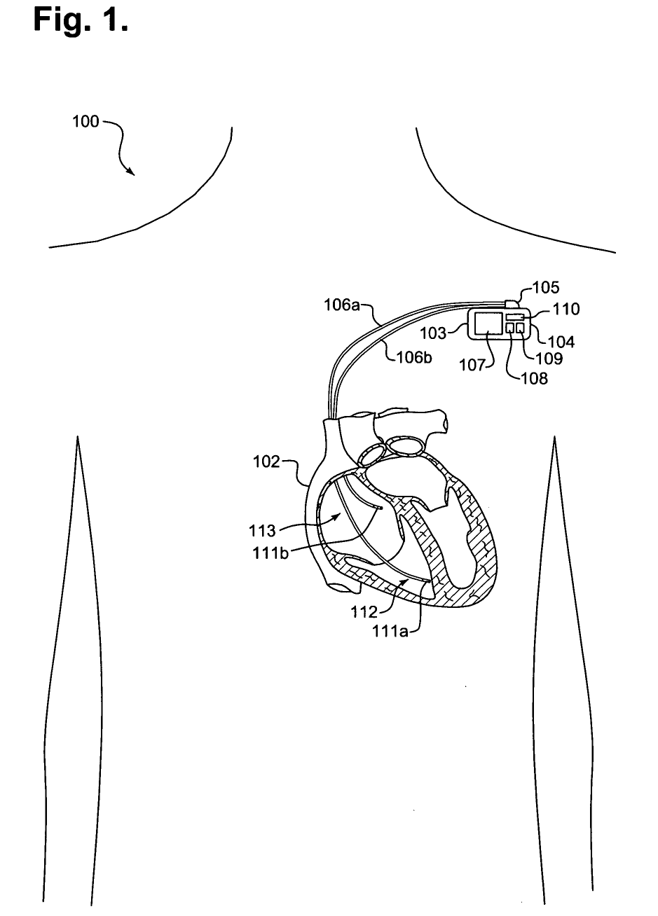

IMDs are frequently used in the care of chronically ill patients to provide in situ therapy and monitoring over an extended time period, whereas EMDs are generally used for short term patient care, often for acute medical disorders. FIG. 1 is a block diagram 100 showing, by way of example, an IMD 103 for use with one embodiment. The IMD 103, such as a cardiac pacemaker, implantable cardiac defibrillator (ICD), cardiac resynchronization device, or similar medical device, is surgically implanted in the chest or abdomen of a patient to provide in situ therapy, such as pacing, defibrillation, cardiac resynchronization, neural stimulation, drug delivery, and physiological data monitoring and collection. Examples of IMDs suitable for use in the described embodiment include the Pulsar Max II, Discovery, and Discovery II pacing systems and the Contak Renewal cardiac resynchronization therapy defibrillators, sold by Guidant Corporation, St. Paul, Minn.

The IMD 103 includes a case 104 and terminal block 105 electrically coupled to a set of therapy leads 106a-b. The leads 106a-b are implanted transvenously for endocardial placement. The IMD 103 is in direct electrical communication with the heart 102 through electrodes 111a-b positioned on the distal tips of each lead 106a-b. By way of example, the set of leads 106a-b can also include a right ventricular electrode 111a preferably placed in the right ventricular apex 112 of the heart 102 and right atrial electrode 111b preferably placed in the right atrial chamber 113 of the heart 102 to enable the IMD 103 to directly collect physiological measures, preferably through millivolt measurements.

The IMD case 104 houses hermitically-sealed components, including a battery 107, control circuitry 108, memory 109, and telemetry circuitry 110. The battery 107 provides a finite power source. The control circuitry 108 controls therapy delivery and patient physiometry monitoring, including the delivery of electrical impulses, or “shocks,” to the heart 102 and sensing of spontaneous cardiac electrical activity. The memory 109 includes a memory store in which physiological signals sensed by the control circuitry 108 can be transiently staged, pending telemetered data download.

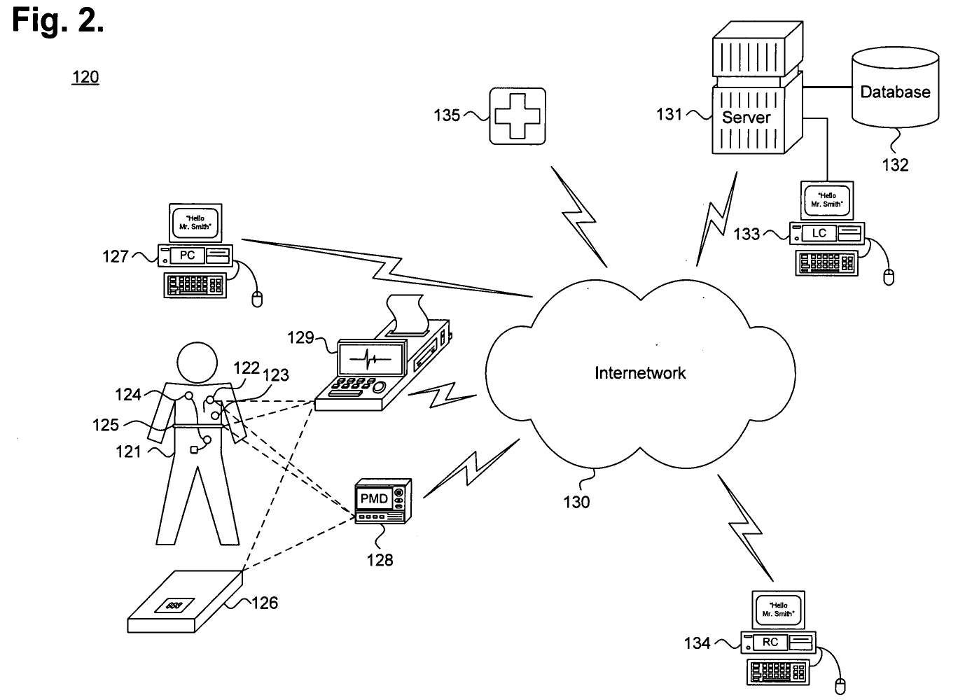

The telemetry circuitry 110 provides an interface between the IMD 103 and an external device, such as a programmer, patient management device, or similar device capable of IMD interrogation. For near field data exchange, the IMD 103 communicates through inductive telemetry signals exchanged through a wand placed over the location of the IMD 103. Programming or interrogating instructions are sent to the IMD 103 and the recorded physiological signals are downloaded. For far field data exchange, the IMD 103 communicates through far field telemetry, such as radio frequency (RF) or optical telemetry, as further described below with reference to FIG. 2. Other data interfaces are possible.

Other configurations and arrangements of leads and electrodes can also be used. Furthermore, although described with reference to IMDs for providing cardiac monitoring and therapy delivery, suitable IMDs include other types of implantable therapeutic and monitoring devices in addition to or in lieu of cardiac monitoring and therapy delivery IMDs, including IMDs for providing neural stimulation, drug delivery, and physiometry monitoring and collection.

### System

Automated patient management encompasses a range of activities, including remote patient management and automatic diagnosis of patient health, such as described in commonly-assigned U.S. Patent application Pub. No. US2004/0103001, published May 27, 2004, pending, the disclosure of which is incorporated by reference. Such activities can be performed proximal to a patient, such as in a patient's home or office; centrally through a centralized server, such from a hospital, clinic or physician's office; or through a remote workstation, such as a personal computer or secure wireless mobile computing device.

Remote patient care can be provided to patients through a combination of implantable or external patient medical devices and a remotely-accessible interface for accessing each patient medical device. FIG. 2 is a functional block diagram showing, by way of example, an automated patient management environment 120. In one embodiment, a patient 121 is proximate to a patient management device (PMD) 128, which is interconnected remotely to a centralized server 131 over an internetwork 130, such as the Internet, or through a public telephone exchange (not shown), such as a conventional or mobile telephone network. In a further embodiment, a remotely-accessible programmer 129, such as a network-capable programmer recorder, can be used by caregivers, such as physicians, nurses, or qualified medical specialists, to interrogate and program patient medical devices. The centralized server 131 can also be remotely interfaced to a patient care facility 135, such as a clinic or hospital, to provide access to emergency medical response or patient care providers. Other patient management devices are possible. The internetwork 130 can provide conventional wired, wireless, or combined forms of interconnectivity. In one embodiment, the internetwork 130 is based on the Transmission Control Protocol/Internet Protocol (TCP/IP) network communication specification, although other network protocol implementations are possible. Other network topologies and arrangements are also possible.

Each PMD 128 is assigned to a patient 121 to provide a localized and network-accessible interface to one or more patient medical devices 122-126, which serve as sources of patient data, either through direct means, such as wired connectivity, or through indirect means, such as induction or selective radio frequency or wireless telemetry based on, for example, “strong” Bluetooth or IEEE 802.11 wireless fidelity “WiFi” interfacing standards. Other configurations and combinations of patient medical device interfacing are possible.

Patient data includes physiological measures, which can be quantitative or qualitative, parametric data regarding the status and operational characteristics of the patient medical device, and environmental parameters, such as temperature or time of day. Other patient data is possible. Patient medical devices 122-126 non-exclusively include both therapy devices and dedicated sensors that measure patient data either as primary or supplemental functions. Patient medical devices for therapy include IMDs 122, such as pacemakers, implantable cardiac defibrillators, drug pumps, and neuro-stimulators; and EMDs 123, such as automatic external defibrillators and continuous positive airway pressure machines. Patient medical devices for monitoring include implantable sensors 124, such as implantable heart and respiratory monitors and diagnostic multi-sensor non-therapeutic devices; and external sensors 125 and 126 that are respectively in contact with or proximate to the patient 121, such as Holter monitors, weight scales, blood oxygen saturation sensors, and blood pressure cuffs. Other types of therapy, physiometric sensing, and data measuring devices, both implantable and external, are possible.

The patient medical devices 122-126 can generate one or more types of quantitative patient data and can incorporate components for delivering therapy, sensing physiological data, measuring environmental parameters, or providing a combination of therapeutic and monitoring functionality. In a further embodiment, qualitative data can also be entered by a patient 121 directly into a patient data source. For example, subjective answers to health-related questions can be input directly into a measurement device, such as a patient-operable personal computer 127, that includes interactive user interfacing means, such as a keyboard and display or microphone and speaker. Such patient-provided data values could also be collected as qualitative patient information. The PMD 128 collects and temporarily stores patient data from the patient medical devices 122-126 for periodic upload over the internetwork 130 to the centralized server 131 and centralized storage in an electronic medical records (EMR) database 132.

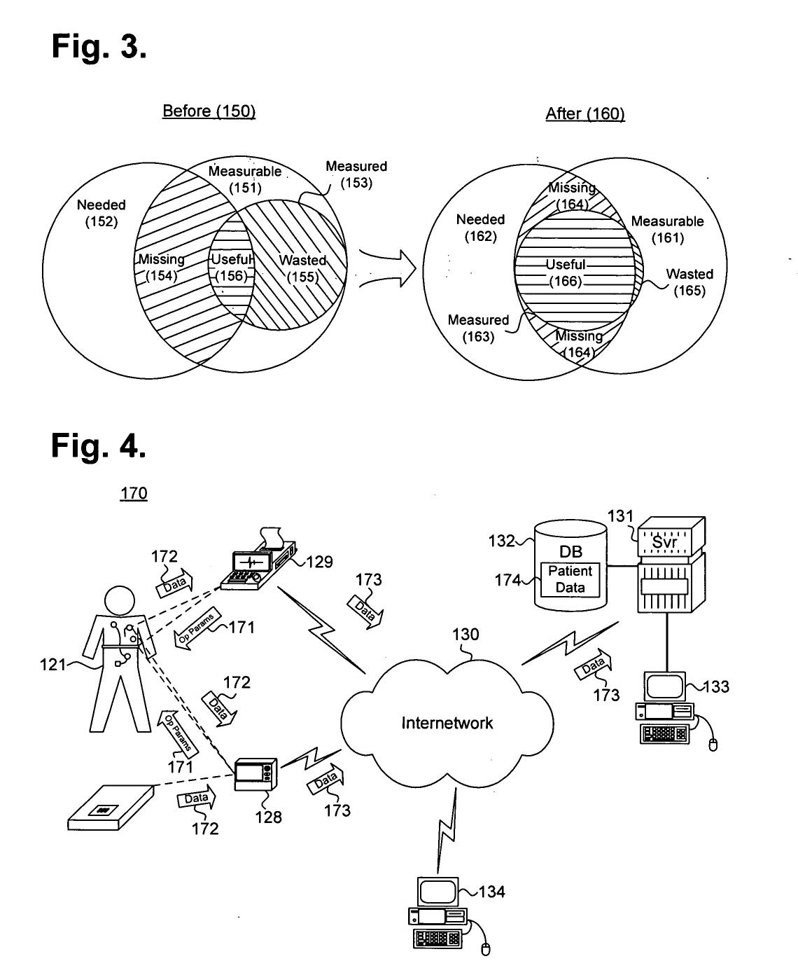

The operational characteristics of the patient medical devices 122-126 can be programmed dynamically to permit flexible selection of and control over collection of patient data, as further described below beginning with reference to FIG. 3. For instance, a caregiver can allocate monitoring resources in the patient medical devices 122-126 to store those measures that are most helpful in following and evaluating a specific patient's well-being, while minimizing the measures stored but not used.

In one embodiment, the patient medical devices 122-126 collect quantitative physiological measures on a substantially continuous or scheduled basis and records the occurrence of events, such as therapy delivery or irregular readings. In a still further embodiment, the personal computer 127 or PMD 128 can record or communicate qualitative quality of life (QOL) measures or symptom assessments that reflect the subjective impression of physical well-being perceived by the patient 121 at a particular time. Other types of patient data collection, periodicity, and storage are possible.

In a further embodiment, the collected patient data can also be accessed and analyzed by one or more caregiver-operable clients, such as locally-configured client 133 or remotely-interconnected client 134. The clients 133, 134 can be used, for example, by clinicians to securely access stored patient data assembled in the database 132 and to select and prioritize patients for health care provisioning, such as respectively described in commonly-assigned U.S. patent application Ser. No. 11/121,593, filed May 3, 2005, pending, and U.S. patent application Ser. No. 11/121,594, filed May 3, 2005, pending, the disclosures of which are incorporated by reference. Although described here with reference to physicians or clinicians, the entire discussion applies equally to organizations, including hospitals, clinics, and laboratories; and other individuals or interests, such as researchers, scientists, universities, and governmental agencies, seeking authorized access to the patient data.

The collected patient data can also be evaluated for the occurrence of one or more medical conditions or health disorders, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 2, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference.

In a further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

Preferably, the centralized server 131 is a server-grade computing platform configured as a uni-, multi- or distributed processing system, and the clients 133, 134 are general-purpose computing workstations, such as a personal desktop or notebook computer. In addition, the PMD 128, centralized server 131 and clients 133, 134 are programmable computing or embedded microprogrammable devices that execute software programs and include components conventionally found in computing device, such as, for example, a central processing unit (CPU), memory, network interface, persistent storage, and various components for interconnecting these components.

### Data Measure Groupings

The quality of health care provided through remote monitoring can be improved by dynamically tuning data collection to better match a particular patient's needs and avoid losing or wasting data measures. FIG. 3 is a set of Venn diagrams showing, by way of example, data measures as grouped before 150 and after 160 dynamic data collection specification through the system 120 of FIG. 2. The careful selection and programming of the operational characteristics of patient medical devices can enable optimal utilization of available resources to measure and collect patient data.

Patient data can be viewed as the set of all data measures recorded and retrieved from patient medical devices. The types of patient data, which are measurable 151 are limited by the types of sensors and related resources available through the patient medical devices. For example, an ICD is incapable of measuring neural activity levels. Moreover, the types of patient data that are needed 152 for patient care may not necessarily coincide with those types of patient data that are measurable 151, possibly due to one or more considerations. For instance, patient data could be missing 154 where physiometric changes are occurring more rapidly than values are recorded due to undersampling below Nyquist limits, or the data measures fall outside the capabilities of the patient medical device to capture. Conversely, patient data might be needed 152, but is missing 154 because the patient medical device is simply not configured to monitor and record those data types. Lastly, of the patient data that is actually measured 153, recorded physiometric measures that are not needed for following a particular patient's health status might be unnecessary and wasted 155, possibly leaving useful patient data 156, that is, measurable 154, needed 152, and measured 153 patient data, as a subset of a larger set of needed and measurable data.

To some degree, the operational characteristics of patient medical devices 122-126 can generally be modified and fine tuned to better address a patient's health condition and a caregiver's need to closely track clinical trajectory. Each patient medical device includes a set of default, factory-specified operational characteristics, which can be later modified by a caregiver through reprogramming or similar operations, as further described below with reference to FIG. 4. Operational characteristics include, for example, the particular types of data to be recorded, the frequency of data collection, whether recorded data will be reduced, and instructions on handling overflow conditions. Other operational characteristics are possible.

The types of useful patient data 156 available before programming of the patient medical devices 150 can be artificially driven by the need to store up to three to twelve months of patient data between interrogations. Remote monitoring, such as through the use of PMDs 128 and programming 129, enable the long-term patient data storage needs to be relaxed through more frequent interrogations and archiving of uploaded patient data. As a result, after programming of the patient medical devices 160, the amount of useful patient data 166, which are both measurable 161 and measured 163, can be fine-tuned to optimize needed patient data 162 and minimize wasted patient data 165. The fewer types of patient data that are missing 164, the better the quality of patient care provided.

### Dynamic Data Collection Specification and Execution

Remote patient management allows a caregiver to use a centralized server 131 as an extension of patient medical devices by storing patient data off-line, thereby freeing limited patient management device resources for tuneable monitoring. FIG. 4 is a functional block diagram showing dynamic data collection specification and execution 170 in the system 120 of FIG. 2. A patient 121, who is a recipient or user of a patient medical device 122-126, is remotely monitored through a PMD 128 or remotely-accessible programmer 129, which are interfaced to the centralized server 131 through an internetwork 130 or other form of connectivity.

Patient medical devices 122-126 that operate autonomously from the patient are generally able to record patient data at any time and under any conditions. However, the recorded patient data accumulated by each patient medical device must be periodically uploaded to free limited onboard resources and to facilitate patient follow-up. When performed over short-term periods, the regular upload of accumulated patient data from the patient medical devices enables the centralized server 131 to serve as a historical extension of the patient data store provided through the patient medical devices. As a result, rather than requiring the patient medical devices to store all recorded patient measures over an extended time period, the frequency of patient follow-up is increased to a daily or weekly, thereby allowing expedited retrieval of the accumulated patient data and enabling the constraints on the limited resources available to each patient medical device to be relaxed.

For example, respiratory measurements should ideally be taken every fifteen minutes for a patient suffering from apnea, particularly at night when the patient is asleep. However, taking four sets of measurements per hour could draw on a significant percentage of the resources available to a patient medical device. In contrast, cardiopulmonary measurements for a patient suffering from atrial fibrillation generally need only be recorded upon the occurrence of an event, rather than periodically. As a result, resources in a patient medical device for a patient presenting with apnea, but no indications of atrial fibrillation, can be reallocated for storing the respiratory measurements and the increased uploading frequency will enable timely retrieval of the respiratory measurements before an overflow condition occurs.

By reallocating patient medical device finite resources, the operational characteristics of each patient medical device 122-126 can be customized by reprogramming the operational parameters to specify the collection of those data measures, which are most relevant to a patients' particular condition that will occur between the more-frequently scheduled patient follow-ups. Operational parameters 171 can be directly defined through a PMD 128 or programmer 129 or indirectly through a client 133, 134, which can facilitate remote programming of patient medical devices in compliance with applicable regulatory requirements. The operational parameters 171 identify the sensor or other device resource for each data measure, specify applicable collection conditions, and allocates storage and other resources for accumulated patient data, as further described below with reference to FIG. 6.

As needed, the operational parameters are downloaded to the patient medical devices and will remain in effect until reprogrammed during a subsequent follow-up session. Meanwhile, accumulated patient data 172 is uploaded with an increased frequency by a PMD 128 or programmer 129 and the uploaded patient data 173 is forwarded to the centralized server 131 on a regular basis for evaluation and storage. The archived patient data 174 remains available to caregivers for use and consideration in follow-up sessions when evaluating clinical trajectory and determining appropriate operational characteristics. In a further embodiment, the archived patient data 174 is stored in a decentralized fashion using a plurality of storage devices or systems, but similarly remains available to caregivers as a historical extension of the accumulated patient data for patient follow-up.

### Method

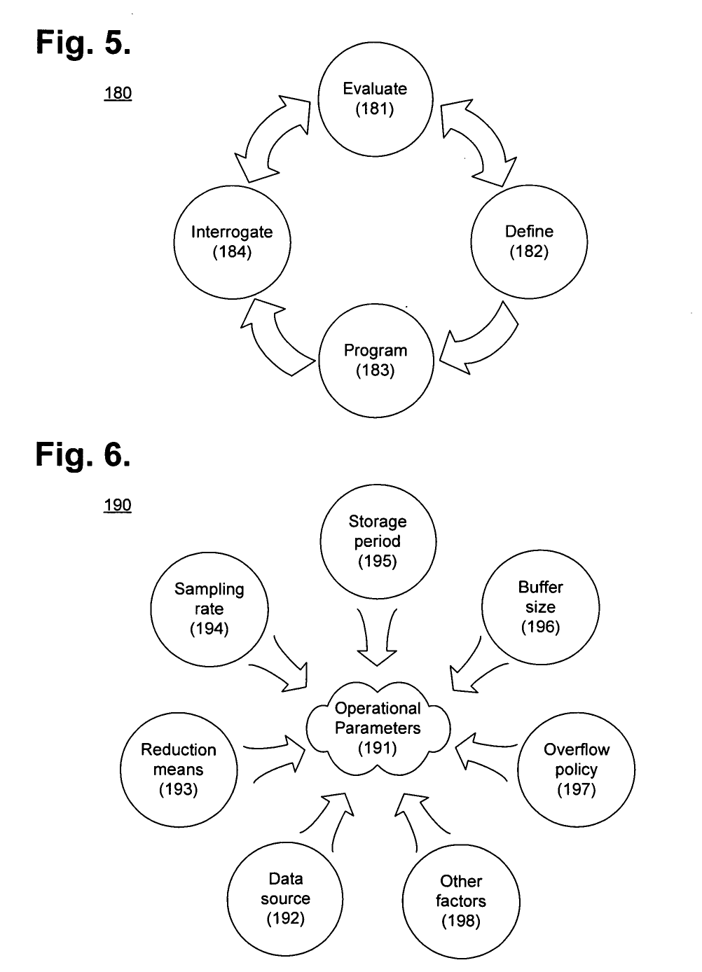

Programming the customized collection of patient data on patient medical devices follows a similar sequence of operations as performed for ordinary long-term patient follow-up, but on an expedited basis and with significantly expanded and more suitable patient data available. FIG. 5 is a process flow diagram showing a method 180 for programming customized data collection for an autonomous medical device, in accordance with one embodiment. Initially, patient data is evaluated (operation 181) to determine a reference baseline and initial patient health status. Operational parameters for patient medical devices are defined (operation 182) and further evaluation (operation 181) can occur as necessary. Once an appropriate set of operational parameters has been formed, the operational parameters are programmed (operation 183) into each patient medical device, which is interrogated (operation 184) during subsequent patient follow-up sessions. The uploaded patient data is again evaluated (operation 181). Further interrogations (operation 184) and evaluations (operation 181) can occur before additional redefinition of the operational parameters may be necessary. Other operations are possible.

### Operational Parameters

Operational parameters specify what, how frequently, and how much patient data will be collected from each patient medical device. FIG. 6 is a block diagram showing, by way of example, operational parameters 191 for specification 190 through the system 120 of FIG. 2. The operational parameters 191 define the sets of features that are programmed into each patient medical device through a PMD 128 or programmer 129. In a further embodiment, the feature sets can be pre-defined remotely and securely distributed over an open communications infrastructure, such as an internetwork 130, such as further described in commonly-assigned U.S. patent application Ser. No. 11/299,980, filed Dec. 12, 2005, pending, the disclosure of which is incorporated by reference.

At a minimum, operational parameters 191 should specify a data source 192 and sampling rate 194 to respectively identify a particular monitoring sensor or device resource and a measurement frequency. Additionally, resources to store each data measurement, such as storage period 195, buffer size 196, and overflow policy 197, should be defined in light of the overall operational profile of a specific patient medical device 122-126. The storage period 195 can be defined in absolute terms by hours, days, weeks, and so forth, or in relative terms, such as number of follow-up sessions. A buffer size 196 specifies the physical memory to be allocated by a patient medical device 122-126 to store a particular data measurement. An overflow policy 197 defines the disposition of patient data once the memory buffer allocated for storage has filled and can include retaining the oldest or newest measures or performing some form of data reduction, such as averaging, over the patient data already stored. Other forms of overflow policy are possible. Finally, if appropriate, a reduction means 193, such as averaging, standard deviation, taking a minimum or maximum, and so forth, can be specified. Other factors 198 may also apply to the definition of the operational parameters 191.

### Scheme Specification

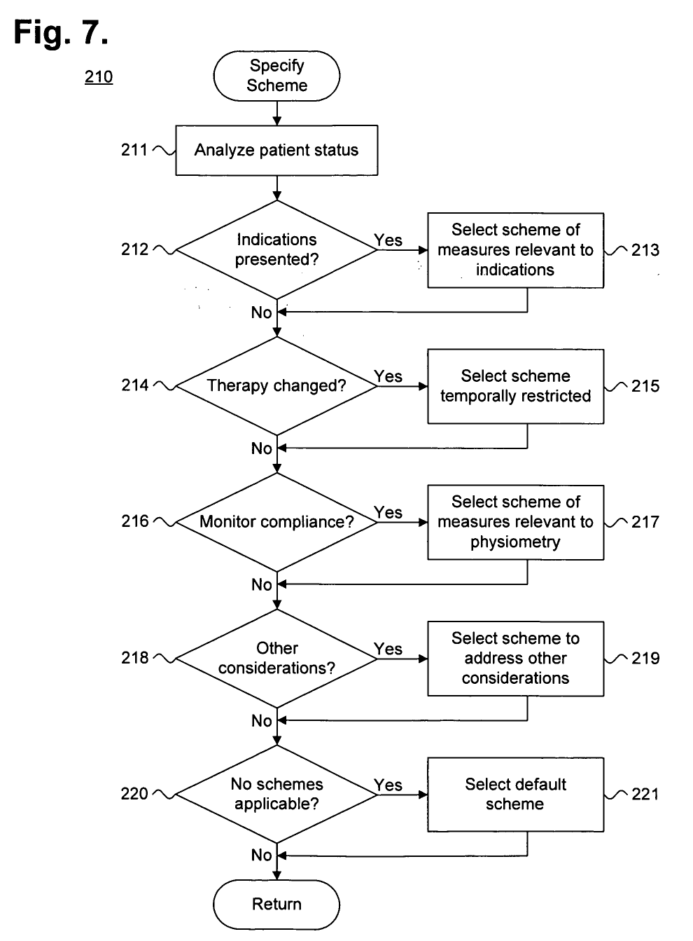

The operational parameters can be specified as part of a patient data collection scheme. FIG. 7 is a flow diagram showing a routine 210 for specifying a scheme for use in the method 180 of FIG. 5. Initially, the patient status is analyzed (block 211) to determine overall patient well-being and clinical trajectory. A scheme can be indication-driven to reduce the storage allocation for parameters that are not relevant to the patient's actual indications. Where particular indications are presented (block 212), a scheme of measures relevant to those indications can be selected (block 213). However, indication-driven schemes are preferably structured to retain the ability to detect changes in patient indications. Similarly, a scheme can be selected in response to a caregiver-specified change in drug therapy (block 214) under which a temporally-restricted scheme might apply (block 215). For example, a change in beta blocker dosage for a cardiac patient might require detailed heart rate data to be collected for a limited period of time following the change. As well, the patient can be monitored under a scheme to ensure therapy compliance (block 216) by selecting a scheme of measures to collect relevant physiometry (block 217). Under this scheme, physiological parameters are monitored at a rate that can detect daily or periodic changes due to drug use as a means to verify patient compliance. Other considerations could apply (block 218) through which appropriate schemes could be selected (block 219), such as a menu of pre-defined schemes for forms or combinations of patient conditions. Finally, if no scheme is applicable (block 220), a default patient data collection scheme can automatically be selected (block 221).

### Trigger Definition

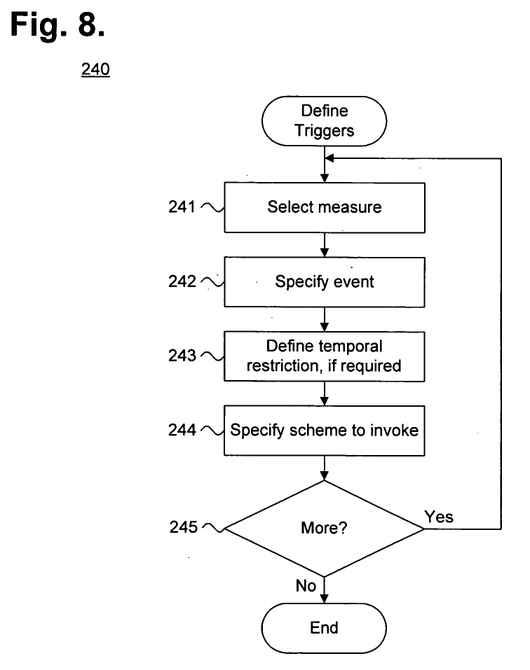

A trigger imparts a change in the operational characteristics of one or more of the patient medical devices 122-126 based on the occurrence of a pre-defined event detected either directly by a patient medical device 122-126 or indirectly during the off-line evaluation of patient data. FIG. 8 is a flow diagram showing a routine 240 for defining triggers for use in the method 180 of FIG. 5. A trigger can apply to one or more selected measures (block 241) based on at least one specified event (block 242). In general, a change in any trended data beyond a pre-defined threshold could lead to more, or less, frequent collection of trended data and related parameters. For instance, the onset of atrial fibrillation could trigger an increase in data collection on V-rates, or a decrease in patient activity level below a pre-defined threshold could trigger an increase in heart rate data collection. A temporal restriction can also be defined if required (block 243) and an associated data collection scheme to be invoked can be specified (block 244). Multiple triggers can be defined (block 245), including enabling recording of data measures; disabling recording of data measures; changing sampling rates; limiting the patient medical device to only performing monitoring; if applicable, setting the patient medical device to provide both therapy and monitoring; and generating an alert.

### Dynamic Memory Control

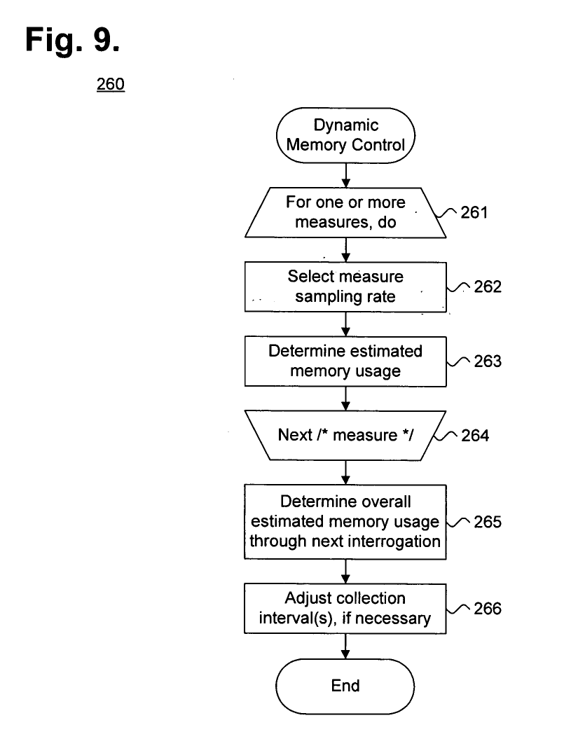

In a further embodiment, a storage and resources allocated for accumulated patient data can be dynamically controlled between data uploads. FIG. 9 is a flow diagram showing a routine 260 for performing dynamic memory control for use in the method 180 of FIG. 5. For one or more of the data measures (block 261), the sampling rate currently in effect is selected (block 262) and the estimated memory usage until the next interrogation is determined (block 263). Memory usage estimates are then determined for each of the data measures under consideration (block 264) for use in determining an overall estimated memory usage for the patient medical device 122-126 (block 265). If necessary, the collection intervals for one or more of the selected data measures are adjusted (block 266) and thereafter put into effect.

In a still further embodiment, the sampling rates and data reduction means could be dynamically controlled to meet a specified collection interval. For example, the appropriate operational parameters could be controlled to ensure that memory would not be filled too quickly, where accumulated patient daily is only collected on a daily basis. Other dynamic control methodologies are possible.

### Data Collection Specifier Structure

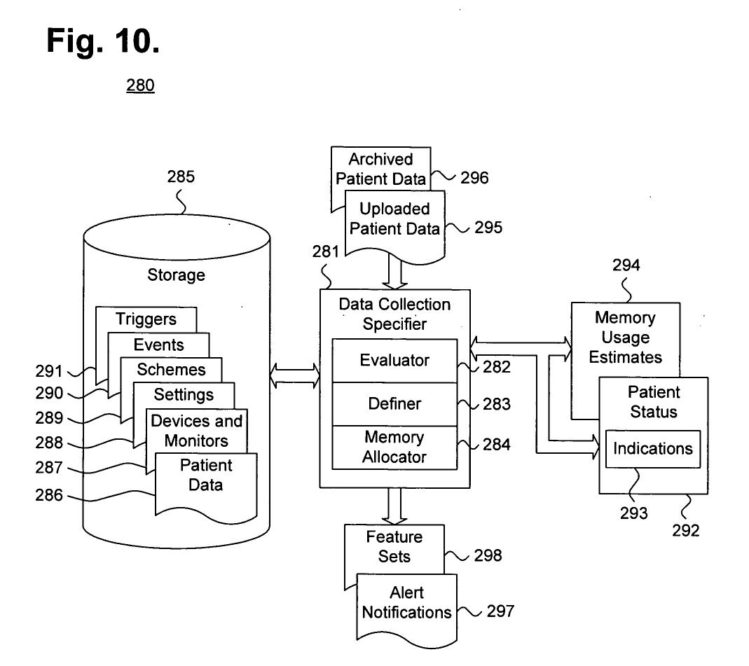

The dynamic programming of data collection by patient medical devices 122-126 can be provided directly a PMD 128 or programmer 129 and indirectly through a client 133, 134, as described above. Generically, these devices can each be referred to as a “data collection specifier.” FIG. 10 is a functional block diagram 280 showing a data collection specifier 281 for use in the system 120 of FIG. 2. In one embodiment, the data collection specifier 281 executes a sequence of programmed process steps, such as described above beginning with reference to FIG. 5, implemented, for instance, on a programmed digital computer or microprogrammable device.

Each data collection specifier 281 includes a storage device 285 and can be configured to store uploaded patient data 286, a listing of all patient medical devices and monitors 287, their settings 288, patient data collection schemes 289, and events 290 and triggers 291. Other types of stored data are possible.

Each data collection specifier 281 includes an evaluator 282, definer 283, and memory allocator 284. The evaluator 282 receives uploaded patient data 295 and archived patient data 296 respectively from the patient medical devices 122-126 and centralized server 131. Other sources of patient data are possible. The evaluator 282 operates on the received forms of patient data to provide assistance to the definer 283 for specifying operational parameters for the collection of data measures. The evaluator 282 forms a patient status 292 that can include one or more indications 293 of a particular medical disorder or condition from which the patient might be suffering. As appropriate, the evaluator 282 can generate alert notifications 297 to the caregiver or patient when an event 290 causes a trigger 291 to be invoked. The definer 283 generates feature sets 298 that provide the operational parameters in a programmable form for each corresponding patient medical device 122-126. Finally, the memory allocator 284 assigns storage and other device resources for transiently staging the patient data recorded by each patient medical device pending upload. In a further embodiment, the memory allocator 284 generates memory usage estimates 294 to re-allocate data collection intervals as required. Other forms of data collection specifier functionality are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for programming customized data collection for an autonomous medical device, comprising:
an evaluator to dynamically identify data measures to be recorded by an autonomous medical device with remote monitoring to evaluate a patient status;
a definer to define one or more operational parameters for the autonomous medical device that specify collection of the data measures, wherein the operational parameters comprise a data source to measure each data measure, collection conditions applicable to the data source, and resources allocated to transiently stage the data measure in the autonomous medical device; and
a programmer to program the operational parameters to effect functional changes in the collection of the data measures to the autonomous medical device, and to regularly interrogate the autonomous medical device to remotely retrieve the data measures recorded under the operational parameters.

2. A system according to claim 1, wherein the data measures are selected from the group comprising physiological measures, parametric data, and environmental parameters.

3. A system according to claim 1, wherein the operational parameters are selected from the group comprising data reduction methodology, sampling rate, storage period, storage buffer size, and overflow policy.

4. A system according to claim 1, further comprising:
a data collection scheme to provide the operational parameters.

5. A system according to claim 4, wherein the data collection scheme is based on conditions selected from the group comprising reducing the resources allocated in the autonomous medical device based on indications presented by the patient, selecting the data measures to track responses by the patient to a change in therapy, and selecting the data measures to monitor therapy compliance by the patient.

6. A system according to claim 4, further comprising:
a menu of data collection schemes.

7. A system according to claim 1, wherein the operational parameters are remotely programmed.

8. A system according to claim 1, further comprising:
at least one trigger for the collection conditions to dynamically alter recording of the corresponding data measure by the autonomous medical device.

9. A system according to claim 8, further comprising:
a processor to perform at least one action associated with each trigger selected from the group comprising enabling recording of the corresponding data measure, disabling recording of the corresponding data measure, changing sampling rate of the corresponding data measure, limiting the autonomous medical device to performing monitoring, setting the autonomous medical device to providing therapy and monitoring, and generating an alert.

10. A system according to claim 8, wherein at least one such trigger comprises detecting changes in the corresponding data measure relative to an absolute or relative threshold value.

11. A system according to claim 1, further comprising:
a memory allocator to estimate usage of memory in the autonomous medical device by the data measures recorded under the operational parameters, and to automatically adjust the collection of the data measures based on the estimated usage.

12. A system according to claim 1, further comprising:
a monitor to monitor overflow of memory in the autonomous medical device; and
an alert generator to generate an alert when overflow is indicated.

13. A system according to claim 1, wherein the autonomous medical device is at least one of an implantable medical device and an external medical device.

14. A system according to claim 1, wherein the data measures recorded under the operational parameters are remotely retrieved using at least one of a patient management device, programmer recorder, and remote patient management system.

15. A method for programming customized data collection for an autonomous medical device, comprising:
dynamically identifying data measures to be recorded by an autonomous medical device with remote monitoring to evaluate a patient status;
defining one or more operational parameters for the autonomous medical device that specify collection of the data measures, comprising:
identifying a data source to measure each data measure;
specifying collection conditions applicable to the data source; and
managing resources allocated to transiently stage the data measure in the autonomous medical device;
programming the operational parameters to effect functional changes in the collection of the data measures to the autonomous medical device; and
regularly interrogating the autonomous medical device to remotely retrieve the data measures recorded under the operational parameters.

16. A method according to claim 15, wherein the data measures are selected from the group comprising physiological measures, parametric data, and environmental parameters.

17. A method according to claim 15, wherein the operational parameters are selected from the group comprising data reduction methodology, sampling rate, storage period, storage buffer size, and overflow policy.

18. A method according to claim 15, further comprising:
providing the operational parameters as a data collection scheme.

19. A method according to claim 18, further comprising:
basing the data collection scheme on conditions selected from the group comprising:
reducing the resources allocated in the autonomous medical device based on indications presented by the patient;
selecting the data measures to track responses by the patient to a change in therapy; and
selecting the data measures to monitor therapy compliance by the patient.

20. A method according to claim 18, further comprising:
displaying a menu of data collection schemes.

21. A method according to claim 15, further comprising:
remotely programming the operational parameters.

22. A method according to claim 15, further comprising:
stipulating at least one trigger for the collection conditions to dynamically alter recording of the corresponding data measure by the autonomous medical device.

23. A method according to claim 22, further comprising:
performing at least one action associated with each trigger selected from the group comprising:
enabling recording of the corresponding data measure;
disabling recording of the corresponding data measure;
changing sampling rate of the corresponding data measure;
limiting the autonomous medical device to performing monitoring;
setting the autonomous medical device to providing therapy and monitoring; and
generating an alert.

24. A method according to claim 22, wherein at least one such trigger comprises detecting changes in the corresponding data measure relative to an absolute or relative threshold value.

25. A method according to claim 15, further comprising:
estimating usage of memory in the autonomous medical device by the data measures recorded under the operational parameters; and
automatically adjusting the collection of the data measures based on the estimated usage.

26. A method according to claim 15, further comprising:
monitoring overflow of memory in the autonomous medical device; and
generating an alert when overflow is indicated.

27. A method according to claim 15, wherein the autonomous medical device is at least one of an implantable medical device and an external medical device.

28. A method according to claim 15, wherein the data measures recorded under the operational parameters are remotely retrieved using at least one of a patient management device, programmer recorder, and remote patient management system.

29. A computer-readable storage medium holding code for performing the method according to claim 15.

30. An apparatus for programming customized data collection for an autonomous medical device, comprising:
means for dynamically identifying data measures to be recorded by an autonomous medical device with remote monitoring to evaluate a patient status;
means for defining one or more operational parameters for the autonomous medical device that specify collection of the data measures, comprising:
means for identifying a data source to measure each data measure;
means for specifying collection conditions applicable to the data source; and
means for managing resources allocated to transiently stage the data measure in the autonomous medical device;
means for programming the operational parameters to effect functional changes in the collection of the data measures to the autonomous medical device; and
means for regularly interrogating the autonomous medical device to remotely retrieve the data measures recorded under the operational parameters.
